(19) United States
(12) Patent Application Publication
(10) Pub. No.: US 2025/0057449 A1
(43) Pub. Date: Feb. 20, 2025

(54) METHODS AND APPARATUS FOR MEASURING ABSOLUTE CONCENTRATION VALUES OF COMPONENTS, BLOOD FLOW AND BLOOD VOLUME IN A TISSUE

(71) Applicant: LUCIOLE MEDICAL AG, Zürich (CH)

(72) Inventors: Dirk BAUMANN, Zürich (CH); Jürg Hans FRÖHLICH, Zürich (CH); Markus Hugo MUSER, Wädenswil (CH)

(21) Appl. No.: 18/705,776

(22) PCT Filed: Aug. 11, 2021
(86) PCT No.: PCT/EP2021/072372

§ 371 Date: Apr. 29, 2024

(51) Int. Cl. A61B 5/145; A61B 5/0205; A61B 5/026; A61B 5/1455

(52) U.S. Cl. CPC A61B 5/14546; A61B 5/0205; A61B 5/0261; A61B 5/1455; A61B 2560/0223

(57) ABSTRACT

Methods and apparatus for determining absolute concentration values of components. a blood flow and/or a blood volume in tissue of an organ, comprising emitting radiation with at least one wavelength in the near-infrared spectrum into the tissue, generating measurement signals from detecting emerging radiation using near-infrared spectroscopy, converting, with an evaluation algorithm, a system matrix and a programmed evaluation unit, a temporal change of the detected intensities of the emerging radiation into absolute concentration values of components, introducing into the tissue an indicator having an absorption maximum in the near-infrared spectrum, and determining a temporal course of concentration values of the indicator in the tissue. Further, a mean transit time mtt is derived from the time course of the concentration values of the indicator and at least one transport function g(t) is used that characterizes blood flow in the tissue, and the blood volume is determined from the time course of concentration values of the indicator or parameters derived therefrom.

### TECHNICAL FIELD OF THE INVENTION

The present invention relates to methods and apparatus for measurement in a tissue, in particular for non-invasive determination of absolute concentration values of components and/or of a blood flow and/or of a blood volume in an organ using an injectable indicator.

### STATE OF THE ART

Known methods for the determination of concentration values of components, blood flow and blood volume in an organ or organ tissue generally comprise a non-invasive measurement, although this does not exclude the injection of a tracer substance.

However, known non-invasive measurement methods for determining concentrations of components in a measurement volume of an organ often cannot determine absolute concentration values but only relative concentration values, i.e., the change in concentration values of one or more components can be detected. Near-infrared spectroscopy (NIRS) is such a non-invasive optical spectrometric method, which is used, for example, for continuous monitoring of oxygen saturation, especially in a living tissue. NIRS is based on the principle that light in the near-infrared wavelength range penetrates biological tissue, is absorbed, and scattered differently by hemoglobin, myoglobin and/or other homologues in the deoxygenated or oxygenated state, and the light attenuation of the transmitted and/or scattered light compared to the light irradiated into the tissue can be detected with a sensor. By means of appropriate algorithms and under certain assumptions, a change in concentration of tissue components, such as oxygenated hemoglobin and deoxygenated hemoglobin, can be calculated from the detected measurement signals. In general, the evaluation of the measurement signals of the biological tissue is based on the diffusion equation and a model obeying the Beer-Lambert law. The Beer-Lambert law describes the attenuation of a radiation intensity in relation to its initial intensity when passing through a medium containing an absorbing substance as a function of the concentration of the absorbing substance and the layer thickness.

In contrast, non-invasive determination of absolute concentration values of components, based on a modified Beer-Lambert law, is difficult and such values can only be approximated under simplifying assumptions. In general, for NIRS measurements in biological tissue, neither an absolute value of the actual absorption nor an actual path length of the light path traveled per defined time period is known. Accordingly, absolute concentration values of chromophores cannot be determined in this way with this model.

U.S. Pat. No. 6,456,862 describes non-invasive determination of a blood oxygen saturation level in tissue using a spectrophotometric sensor in the near-infrared range, whereby absolute concentration values of oxyhemoglobin and deoxyhemoglobin can be determined. The sensor emits a light signal of first, second, and third wavelengths into a tissue and detects a light signal after passing through the tissue. The attenuation of the light signal is described as the sum of the attenuations based on the deoxyhemoglobin and oxyhemoglobin components and the scattering within the tissue. For this method, calibration of the sensor with respect to the tissue under consideration is required to account for light signal attenuation due to light scattering, due to absorption by solid tissue components such as bone and/or water, and/or due to varying sensor properties during evaluation. Empirical data may be taken into account for calibration, or arterial oxygen saturation may be determined using pulse oximetry as a reference sensor to determine venous oxygen saturation.

In addition to the relative concentration changes or the absolute concentration values of components in a tissue of an organ, blood flow and/or blood volume can be considered as diagnostic parameters, which contain information about the blood perfusion and functionality of the tissue under consideration. Near-infrared spectroscopy (NIRS) also has been used for a long time for the non-invasive determination of these diagnostic parameters. This allows, based on the determined concentration changes of components of the considered organ tissue, to draw conclusions about regional oxygen metabolism. Use of NIRS is known for monitoring cerebral blood flow (CBF) and cerebral oxygenation patterns, i.e., to determine static and dynamic properties of cerebral blood and blood flow, respectively.

For the determination and monitoring of cerebral blood flow, a method is known that combines non-invasive measurement with a method in which a tracer substance is injected intravenously. The tracer substance may be, for example, largely inert indocyanine green (ICG), the distribution of which is largely confined to the intravascular compartment. Indocyanine green has an absorption maximum at about 805 nm and thus lies in the spectrum of near-infrared light. ICG is very rapidly and almost completely bound to serum albumin in blood plasma, shows very high NIR absorption in this state, and is absorbed by the liver, allowing repeated prompt measurements. ICG is used for various investigations of liver function and/or to calculate cardiac output from recirculation times.

Starting from a bolus injection of indocyanine green, its absorption in a cerebral vascular system under consideration can be determined invasively and/or non-invasively, e.g., by means of NIRS. The measurement data then can be further processed to determine, for example, cerebral blood flow.

U.S. Pat. No. 7,529,576 describes a device for measuring blood flow and blood volume in an organ, in particular cerebral blood flow (CBF), using an injected, largely inert indicator. Using a sensor known per se, the device detects the portion of irradiated near-infrared radiation emerging from the organ tissue, which contains a pulsatile portion and a non-pulsatile portion. An evaluation algorithm calculates the blood volume in the organ as a quotient of the concentration from the non-pulsatile component related to the organ tissue and the concentration from the pulsatile component of the injected indicator related to the blood volume in the organ, taking into account an inflow function characterizing the organ perfusion. The value of the blood volume related to a determined mean transit time permits calculation of the blood flow in the organ. However, one shortcoming of this method is that due to the superficially attached sensor, the influence of layers located between the measurement volume and the point of entry and/or exit of the measurement signal distorts and weakens the measurement signal, and this effect is not taken into account in the evaluation.

U.S. Pat. No. 6,223,069 describes a method for determining cerebral blood flow and blood flow of extracerebral organs, whereby the determination of a flow of an intravenously injected bolus of a tracer substance with absorption properties in the near-infrared spectrum is carried out simultaneously in both brain hemispheres using near-infrared spectroscopy and in the arterial blood of the systemic circulation by means of pulse densiometric arterial dye measurement. The evaluation algorithm developed to evaluate the measured values is based on deconvolution of the arterial and cerebral flow kinetics to calculate the transcerebral transport function. From those kinetics, a blood flow index can be determined, which is directly proportional to the cerebral blood flow.

Previously known methods and apparatus for non-invasive measurement of blood flow, in particular blood volume and/or absolute concentration values of components in a tissue or organ using an injected indicator often are technically difficult to implement and, moreover, time-consuming. In addition to inaccuracies created by employing simplifying assumptions with respect to evaluation of measurement data of conventional

NIRS measurements for behavior of the injected indicator during the first rise of the input signal, a further problem arises from a non-constant measurement background with varying absorption properties.

Up to now, a simplified interpretation of the measurement data often assumed that no change of the background absorption occurs during a NIRS measurement. With this assumption, differential absorptions may be calculated from a modified relation of the Beer-Lambert law. Assuming an idealized constancy of the background, its influence, e.g., due to tissue geometry and its composition as well as its absorption behavior, is summarized to a factor G. In real systems and under real conditions, differential absorptions may be calculated from this modified relation of the Beer-Lambert law. In real systems and under real conditions, however, the background absorption does not remain constant. In particular, the water concentration for a cerebral measurement volume and/or the cerebral intracranial pressure are not constant, thereby introducing inaccuracies in the evaluation of the measurement signals of an NIRS and the determination of concentration values, blood volume and blood flow of cerebral tissue.

In view of the disadvantages of previously known methods and apparatus, it would be desirable to provide methods and apparatus for determining absolute concentration values of components in a tissue. More particularly, it would be desirable to determine such absolute concentration values in cerebral tissue, as well as blood flow, and more particularly cerebral 10 blood flow and blood volume, with higher accuracy than heretofore possible.

It further would be desirable to provide methods and apparatus for evaluating detectable measurement signals that are decoupled or largely free from simplifying assumptions and the influence of conditions. Preferably, such apparatus may be used or implemented non-invasively and combined with an indicator injection with justifiable technical effort. Furthermore, such apparatus may be used to conduct invasive measurements, for example, with sensor units or optodes disposed under a body surface.

### SUMMARY OF THE INVENTION

According to the present invention, methods and apparatus are provided that are expected largely to eliminate the influence of the background or changes in background absorption when determining absolute concentration values of components of a tissue by accounting for variations in the background and concentration of tissue water. In this manner, changes in the measurement conditions are verifiable, with greater accuracy and higher significance of the results.

Likewise, when determining blood flow and blood volume, the influence of an employing an indicator is reduced. Whereas in previously known systems, the determination of blood flow and/or blood volume by application of an indicator assumed that an indicator bolus has the shape of a rectangular pulse, the methods and apparatus of the present invention avoid such simplifying assumptions. In particular, due to dispersion effects, pulsation, and other factors to be taken into account, a rectangular shape does not exist in real terms and that simplifying assumption leads to distorted quantification. In reality, the increase in an indicator concentration in the considered volume takes the form of a fuzzy peak. In accordance with the principles of the present invention, the influence of the real increase of the indicator concentration on the accuracy of the result of the measurement to be obtained can be largely eliminated.

According to the invention, methods and apparatus for determining absolute concentration values of components, blood flow and/or blood volume in a tissue of an organ are provided. In one embodiment, a sensor emits radiation with at least one wavelength in the near-infrared spectrum into a tissue of an organ. The emerging radiation is detected by the sensor, and measurement signals are generated employing near-infrared spectroscopy. Those measurement signals responsive to detected intensities of radiation emerging from the tissue are input to a programmable evaluation algorithm in a programmed evaluation unit to compute a temporal change of the detected intensities of the emerging radiation, and thereby and by using a system matrix compute absolute concentration values of components.

In accordance with one aspect, an indicator having an absorption maximum in the near-infrared spectrum is introduced during acquisition of the near-infrared spectroscopy data, and a temporal course of concentration values of the indicator in the organ tissue is determined. A mean transit time, mtt, is derived from the time course of the concentration values of the indicator and using at least one transport function g(t) that characterizes blood flow in the organ tissue, the blood volume is determined from the time course of concentration values of the indicator or parameters derived therefrom.

Further details of the invention will become apparent from the following description of preferred embodiments of the invention, which are shown in the accompanying drawings. Further advantages of the invention can also be seen from the description, as well as suggestions and proposals as to how the objects of the invention could be modified or also further developed within the scope of what is claimed.

### BRIEF DESCRIPTION OF THE DRAWINGS

FIG. 1 is a schematic of the inventive apparatus for determining absolute concentration values and/or blood flow in a tissue under consideration, using cerebral tissue as an example;

FIG. 2 is a schematic of a programmed evaluation unit suitable for implement the algorithms of the present invention; and

FIG. 3 is a schematic of the method of the present invention, which is programmatically implemented in an evaluation unit to determine the cerebral blood flow from an input signal.

### DETAILED DESCRIPTION OF THE INVENTION

In accordance with the principles of the present invention, absolute values of physiologic components may be calculated using a sensor that emits radiation with a wavelength in the near-infrared spectrum and detects emerging radiation to generate measurement signals using near-infrared spectroscopy. A bolus of indicator is introduced intravascularly during the near-infrared spectroscopy, which can be used to iteratively determine inflow and outflow functions using a programmed evaluation unit. The indicator, which may be a dye, is introduced using an indicator injector device, e.g., syringe or infusion device, into the vascular system, which is known per se.

More specifically, the concentration of the indicator bolus in the considered volume may be described by an inflow function, and an outflow function. Because these functions are not known or measurable, certain assumptions are made to permit iterative determination of these functions. For this purpose, according to the invention, a programmable evaluation algorithm is provided that takes into account an outflow of the injected indicator from the considered area or volume during the initial rise of the measurement signal.

The method according to the invention for determining absolute concentration values of components and/or blood flow and/or blood volume in tissue of an organ comprises, among others:
Emitting radiation with at least one wavelength in the near-infrared spectrum into the tissue and detecting the emerging radiation intensity by means of near-infrared spectroscopy as measurement signals responsive to detected intensities of radiation emerging from the tissue;
Converting a temporal change of the detected intensities of the radiation emerging from the tissue of the organ into absolute concentration values of the components using a system matrix and an evaluation algorithm that is executed programmatically in an evaluation unit;
Applying an indicator comprising a dye having an absorption maximum in the near-infrared spectrum during near-infrared spectroscopy and determining a time course of the concentration values of the indicator in the tissue;
Deriving a mean transit time mtt from the time course of the concentration values of the indicator and using at least one transport function g(t) that characterizes blood flow in the tissue, and
Calculating blood flow and/or blood volume from the time course of concentration values of the indicator and/or derived parameters.

In one embodiment of the method, the system matrix initially may be calibrated using known values of concentrations of the components in the tissue such as of the blood, background, and/or water, which concentration values are obtained from in vivo measurements of healthy patients, or of healthy tissues, and/or boundary conditions to be defined for limiting the concentration values of components in the tissue.

According to one embodiment of the method, the method is arranged to determine from the emitted and detected radiation intensities of the emitted radiation, in particular from the measurement signals, absolute concentration values of at least one of the components comprising hemoglobin, deoxyhemoglobin, water, background and/or indicator, as well as the blood volume and/or blood flow in the tissue of the organ.

In particular, radiation of at least one wavelength in the near-infrared spectrum, and more preferably, radiation of at least four wavelengths, is emitted into the tissue of an organ at a first position. A measurement signal is detected by a sensor disposed at a second position, spaced from the first position, which represents the emitted and detectable radiation intensity or a component of the emitted radiation. Preferably, each of the at least four wavelengths may be selected such that each of the components to be detected has a local absorption maximum or at least an even as possible absorption of all components. The detected measurement signal may be transmitted as an input signal to the programmed evaluation unit, e.g., one or more processors, which programmatically execute the evaluation algorithm with the following steps:
a) Acquire the measurement signals that transmitted to the evaluation unit, based on an emitted and detected portion of the emitted radiation with at least one wavelength in the near-infrared spectrum;
b) Determine absolute concentration values of the components in the tissue of the organ of at least hemoglobin, deoxyhemoglobin, background, and/or water;
c) Determine the time course of the concentration of the injected indicator in the tissue of the organ from the measurement signals;
d) Iteratively determine an inflow function i(t) and an outflow function o(t), which characterize blood flow in the tissue of the organ, using a transport function g(t) with a determinable mean transit time mtt until a termination criterion is reached;
e) Fit the iteratively determined inflow function i(t) and the iteratively determined outflow function o(t) using a lognormal function or another function type representing tissue transit system;
f) Calculate blood volume in the tissue of the organ using one of the time function curves determined in step c) or e); and
g) Calculate blood flow in the organ as a quotient of the blood volume in the tissue of the organ calculated in step f) and mean transit time mtt determined in step d).

Based on the detectable near-infrared radiation emerging from the organ tissue, i.e., a detected intensity signal, a value of an optical density (OD) can be determined. In general, optical density or extinction is a measure of the attenuation of a radiation as it passes through a medium, which is due to absorption, scattering, diffraction, and/or reflection. The value of the optical density can be determined as the negative logarithm of a transmission as a function of the emitted wavelength.

Absolute concentration values of the components of the organ tissue as well as of the background related to the measurement volume then may be determined using the preferably calibratable system matrix. The system matrix describes the imaging property of NIR spectroscopy, taking into account optical path lengths at different wavelengths, extinction coefficients of the considered components and molecular weights of individual components, for example, of hemoglobin and water, etc. The system matrix may be used in the solution of a system of equations, such that absolute concentration values of the components may be determined from detected measurement signals.

According to one embodiment, for a preferably one-time calibration of the system matrix, the background of the tissue of the organ to be considered can be considered. The background may be composed of several components in several combination thereof, e.g., tissue, fat, bone, etc., for each of which the extinction coefficient is used as specific substance constant. More generally, the extinction coefficient depends on the wavelength of the emitted radiation and on the temperature prevailing during the measurement. Extinction coefficients of the components of the background may be taken from clinical data and/or from the literature for known concentration values of blood, or rather hemoglobin and/or water, etc., in proportion to the expected presence of such constituents in the background. By means of the extinction coefficients known per se, calibration of the NIR spectroscopy or rather the system matrix may be performed. Accordingly, calibration is an integral part of the evaluation algorithm for the determination of absolute concentration values of the components of the tissue of the organ.

In addition to the absorption properties of the inhomogeneous background, the optical density of the tissue under consideration is taken into account when setting up the system matrix, for example, the cerebral optical density. The solution space of the system matrix may be restricted by bringing the determined optical densities of the tissue at different wavelengths and the weighted sum of the concentration values of the components into a definable relationship.

Using the detectable time-varying optical density(s) and using the system matrix, absolute concentration values of the components of the organ tissue may be calculated.

According to one aspect of the invention, the method may include determining blood volume and/or blood flow using indocyanine green as the injectable indicator. However, use of this indicator is not limiting. In such an embodiment, the concentration of the injected indicator in the organ tissue under consideration will depend on the concentration at which the injected indicator enters the organ tissue with the inflowing blood and the volume of blood flowing through the organ tissue. The concentration of the injected indicator in the organ tissue changes due to distribution kinetics and degradation, so that the blood flowing out of the organ tissue has a different concentration of the indicator or a different time course of concentration than that of the inflowing blood. After application of the indicator, for example in the form of an indicator bolus, the indicator reaches the tissue to be examined after a certain time, the so-called transit time mtt, through the blood flow and perfuses into the existing capillary network or vascular system. From a determinable concentration-time curve of the injected indicator, a distribution volume of the same and furthermore the blood volume and/or blood flow in the tissue of the organ under consideration can be determined, whereby real effects can be taken into account in the evaluation.

According to one embodiment of the method, the blood flow in the tissue can be determined from the time course of the determinable concentration values of the indicator in the organ or tissue of the organ. For this purpose, inflow function i(t) and outflow function o(t) may be iteratively determined. Inflow function i(t) describes the proportion of the change in concentration of the injected indicator in the measurement volume under consideration, for example the cerebral tissue, caused by the inflowing blood volume. Outflow function o(t) describes the proportion of the change in concentration of the indicator in the measurement volume caused by the outflowing blood volume.

In the iterative determination, each step involves an approximation to inflow function i(t), which can be expressed as a function of the time course of the concentration of the indicator cICG(t) in the tissue of the organ and outflow function o(t):

$$cICG ( t ) = {\int }_{- ∞}^{r} ( i ( τ ) - o ( τ ) ) d τ$$

Outflow function o(t) is determinable by deconvolution of a convolution integral of inflow function i(t) and transport function g(t) with * as convolution operator:

$$o ( t ) = i ( t ) * g ( t )$$

As a starting parameter of the iteration for the determination of inflow function i(t), an estimated or determinable mean transit time mtt is chosen, which is also called turnaround time. The initial value of mean transit time mtt may be determined by integration of the time course of the concentration values of the injected indicator cICG(t), fitted with a lognormal distribution or another function type representing tissue transit system, i.e., representing the transit behavior of the blood flow through the tissue.

During iterative determination of inflow function i(t) and outflow function o(t), a time variable t is set to zero for the time of the indicator increase in the measurement volume. Furthermore, inflow function i(t) and outflow function o(t) are set to zero for t<0 to ensure causality between the time of indicator application and a measurable indicator increase.

Inflow function i(t) and outflow function o(t) are determined for the assumed mean transit time mtt using transport function g(t). A time course of transport function g(t), generally a single-peaked, asymmetric distribution, can be approximately described by a lognormal function, for example. Here, mean transit time mtt and consequently transport function g(t), as well as the associated inflow function i(t) and outflow function o(t), are varied until a termination criterion to be determined is reached.

Inflow function i(t) at a time t may be represented according to a balance equation from the time course of the concentration values of indicator cICG(t), e.g., indocyanine green ICG, and outflow function o(t), according to:

$$i ( t ) = \frac{d}{d t} ( cICG ( t ) ) + o ( t )$$

For the calculation of inflow function i(t), first the concentration of indicator cICG(t) in the considered tissue is determined from a determinable time course of the optical density OD(t) of the component indocyanine green as the indicator, extinction coefficient ε_{ICG}, and a covered optical path length β of the light, according to:

$$cICG ( t ) = ( OD ( t ) - O D ( t = 0 ) ) / ( {ε}_{ICG} \cdot β )$$

Outflow function o(t) at a time t can be formulated as a convolution integral of inflow function i(t) and transport function g(t). That is, a deconvolution of the convolution integral may be used to determine inflow function i(t) and/or outflow function o(t), where τ is a small constant time interval, i.e., an increment:

$$o ( t ) = i ( t ) * g ( t ) = \int i ( τ ) g ( t - τ ) d τ$$

Transport function g(t) can be formulated according to one of the approaches known per se as a function of the transit time mtt. A suitable approach is the lognormal function:

$$g ( t ) = \frac{1}{\sqrt{2 πσ t}} \cdot ex p ( \frac{ln \frac{t}{m t t} + \frac{{σ}^{2}}{2}}{2 {σ}^{2}} )$$

Here σ is a constant shape parameter whose value is a real number greater than 0. The shape parameter σ may be determined on the basis of empirical values and σ largely represents the width of an assumed residence time distribution. As the value of σ decreases, the symmetry of the lognormal distribution increases.

In a subsequent step, deconvolution of the established convolution integral is performed to determine inflow function i(t) as well as outflow function o(t), based on the determinable time course of the concentration of indicator cICG(t). Here, the influence of a recirculation on the temporal change of the concentration of indicator cICG(t) in the considered measuring volume, and thus also its influence on inflow function i(t) and outflow function o(t), is eliminated by fitting a lognormal function.

In a subsequent step, it can be checked whether the previously determined function curves of inflow function i(t) and outflow function o(t) are plausible. Accordingly, the termination criterion for determining mean transit time mtt may be defined by a plausibility criterion that is related to inflow function i(t) and outflow function o(t).

In one embodiment, the plausibility criterion can be defined by a distance from a centroid of inflow function i(t) and a centroid of outflow function o(t), such that the distance corresponds approximately to mean transit time mtt.

Alternatively, the plausibility criterion can be defined by a ratio of an area under inflow function i(t) and an area under outflow function o(t), which are in a definable ratio to each other. For example, the ratio can be 1:1.

In the case in which the function curves of inflow function i(t) and outflow function o(t) fulfill one of the plausibility criteria, i.e., a termination criterion is reached, variation of the transit time mtt is terminated and the procedure for determining the blood volume and/or the blood flow in the organ tissue under consideration is continued with the determined function curves of inflow function i(t), outflow function o(t), and the determined mean transit time mtt. In the event that the functional curves of inflow function i(t) and outflow function o(t) do not meet any of the plausibility criteria, mean transit time mtt can be increased or decreased by increment τ to again perform the iteration with the steps described above, which begins with the correspondingly changed mean transit time mtt as the start parameter.

According to one aspect of the method, blood volume in the organ tissue can be determined by the areas under inflow function i(t) and outflow function o(t), based on the assumption that these areas are proportional to blood volume BV, e.g., cerebral blood volume CBV. Here, the integration limits for area determination are selectable depending on the width of the curves of i(t) and o(t).

As an alternative to determining blood volume using an integration of inflow function i(t) and outflow function o(t) to determine the corresponding areas, the time course of the concentration values of indicator cICG(t) can be used. The time course of the concentration values of indicator cICG(t) can be approximately described by an exponential function, using regression, in a time interval t1>0 up to a final value t2>t1. The exponential function then may be extrapolated back to a specific time, e.g., t=0, i.e., the time of the indicator injection.

Accordingly, from the determined time course of the concentration values of indicator cICG(t), calculation of the indicator concentration cTissue in the considered organ tissue immediately after the injection may be determined with the formula:

$$c T i s s u e ( t ) = cICG ( t \to 0 )$$

Blood volume BV of an organ, e.g., cerebral blood volume CBV, is calculated as a quotient of indicator concentration cTissue related to the considered tissue, the known indicator concentration in blood, cBlood, and tissue density, ϑTissue, according to:

$$B V = c T i s s u e / ( cBlood \cdot Tissue )$$

The indicator concentration in blood cBlood may be determined from the total blood volume BV and the injected amount of indicator.

Blood flow BF in the considered organ may be determined as a quotient of blood volume BV, e.g., cerebral blood flow CBF, and mean transit time mtt according to the following formula:

$$B F = B V / m t t .$$

As described below, the present invention includes apparatus provided and/or programmed to implement the method. In a preferred embodiment, the apparatus includes an evaluation unit programmatically arranged to execute an evaluation algorithm, a unit comprising electrical and/or optical components and at least one sensor arrangement for supplying radiation having at least one wavelength in the near-infrared spectrum. The sensor unit comprises optical components with at least one detector device for detecting an outgoing portion of the emitted radiation.

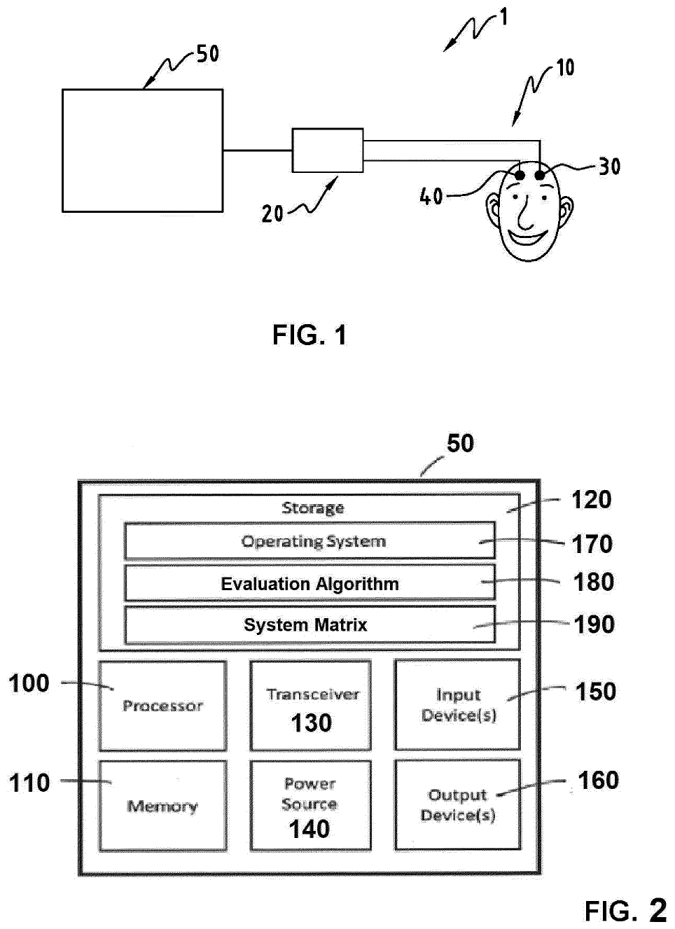

Referring now to FIG. 1, apparatus 1 is described for determining absolute concentration values and/or for determining the blood volume and/or the blood flow in a cerebral tissue. For this purpose, apparatus 1 includes a non-invasive measuring device and method that is coupled with an indicator application. Sensor arrangement 10 is releasably attachable to a body surface of a patient. Alternatively, sensor arrangement 10 may be inserted through the patient's body surface for invasive determination of absolute concentration values.

Sensor arrangement 10 comprises electrical and optical components, for example combined in unit 20, as well as at least one sensor unit 30 to emit radiation with at least one wavelength in the near-infrared spectrum and at least one detector unit 40 to detect radiation emerging from the body tissue. Radiation, preferably in multiple different wavelengths in the near-infrared spectrum, is emitted into the body tissue from an external light source or from a light source located directly in unit 20 or in sensor unit 30, such as multiple light or laser diodes. The at least one wavelength of the emitted radiation is matched to the injected indicator, which may be, for example, indocyanine green ICG. When indocyanine green ICG is injected as an indicator, one of the wavelengths may be in a range of 780 to 900 nm, and preferably at about 808 nm. For the determination of optical densities of multiple components in the measurement volume, the use of at least four wavelengths is desirable.

Sensor arrangement 10 comprises the at least one detector unit 40, preferably a first detector unit and a second detector unit (not shown), which are spaced apart an optimized distance from each other. Here, optimization may be understood with respect to the penetration depth of the emitted radiation into the body tissue and/or with respect to a signal-to-noise ratio.

The first and second detector units (not shown) are set up to detect a portion of the radiation emerging from the tissue as measurement signals, which signals are transmitted to evaluation unit 50. Transmission of measurement signals may occur in real time by cable or wirelessly. Evaluation unit 50 may comprise electronics, e.g., one or more processors, and/or light sources and preferably is programmed to execute programs. The one or more processors may communicate with each other either via a wired bus or wirelessly. In the case of wireless communication, remote access is possible at any time.

Referring now to FIG. 2, an exemplary embodiment of evaluation unit 50 is described. Evaluation unit 50 includes a computer, e.g., laptop, desktop, or tablet that is programmed with the evaluation algorithm software as described herein, and includes at least one processor 100, memory 110, non-volatile storage 120, optional transceiver 130, power source 140, and one or more input devices 150 and output devices 160.

Processor 100 may be a conventional multi-core processor, such as an Intel CORE i5 or i7 processor. Memory 110 may comprise volatile (e.g., random-access memory (RAM)), non-volatile (e.g., read-only memory (ROM)), flash memory, or any combination thereof.

Optional transceiver 130 may receive and/or transmit non-invasive measurement information to and from other components in the system, such as a remote monitoring station, and/or a controller for injecting an indicator dye, using any well-known communication infrastructure facilitating communication over wired or wireless connection, such as any IEEE 802 standard. Power source 140 preferably connects to a standard wall outlet and/or may include a battery. Nonvolatile storage 120 preferably includes removable and/or non-removable storage, such as, solid-state disk memory or magnetic hard drive.

Input device 150 may be one or more devices coupled to, or integrated into, evaluation unit 50 for inputting data to the evaluation unit, and may include, for example, a keyboard or touchscreen, a mouse and/or a pen. Input device 150 may be used to input data used in populating the system matrix, as well as patient specific information into the evaluation unit, e.g., height, age, skin tone, gender, and identity. Output device 160 may be any suitable device coupled to or integrated into evaluation unit 50 for outputting or otherwise displaying data, such as video screen, printer, or plotter. Output device 160, further may include a speaker or alarm bell that may be activated if one or more of the measurement signals falls below a clinically significant threshold indicating patient distress.

Still referring to FIG. 2, operating system 170 and evaluation algorithm 180 are stored in non-volatile storage 120. Operating system 170 includes the operating system for the evaluation unit, e.g., Microsoft Windows or Linux, as well as the necessary drivers for the input and output devices.

Evaluation algorithm 180 may be personalized for a specific patient based on inputs provided through input devices 150, or alternatively may include machine learning and search algorithms for analyzing the input patient specific data to modify the data in system matrix 190 to generate real-time extinction values suitable for the patient being monitored. Evaluation algorithm 180 also may include programming for communicating with and adjusting the input sampling rates for inputs received from sensor arrangement 10, and further for generating output to be displayed on output device 160.

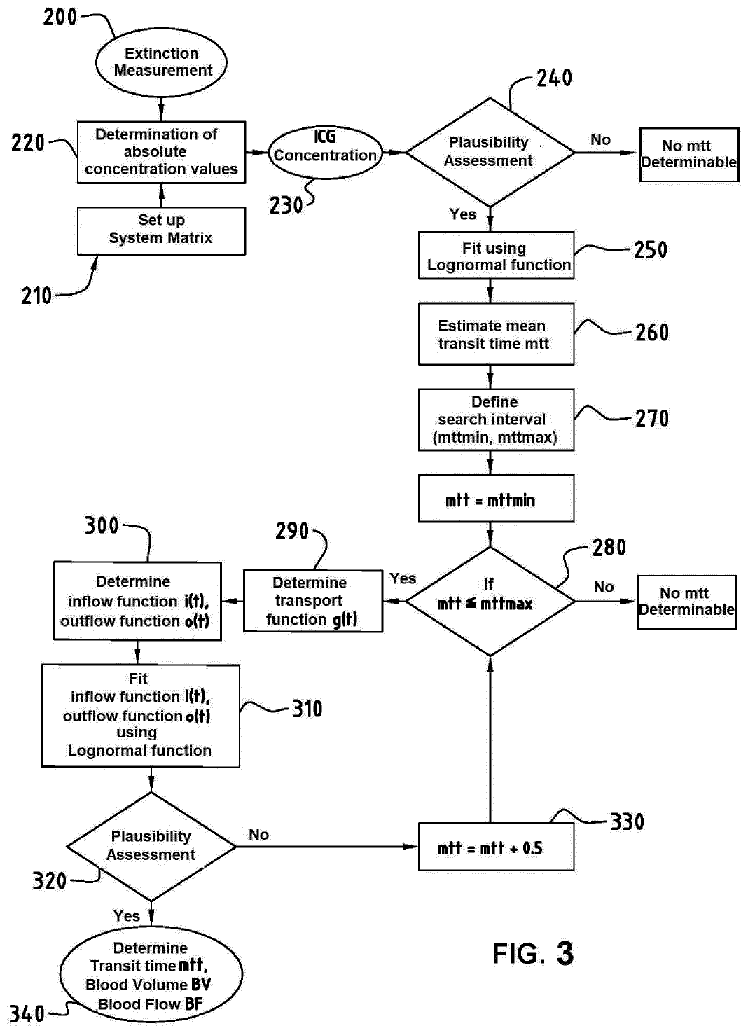

Evaluation algorithm 180 includes programmed instructions for generating absolute values of blood volume and blood flow, particularly cerebral blood volume and blood flow, using noninvasive measurement signals obtained from sensor arrangement 10, as described herein. Specifically, the evaluation algorithm may employ an iterative process for determining, using noninvasively-measured patient physiologic data, absolute concentration values of specific blood constituents, e.g., oxyhemoglobin and deoxyhemoglobin, blood flow and/or the blood volume of a tissue of an organ according as described in the flow chart of FIG. 3.

With respect to FIG. 3, an exemplary flow chart is described that corresponds to programmed evaluation algorithm 180 stored in non-volatile storage 120 of evaluation unit 50.

By means of sensor arrangement 10 shown in FIG. 1, extinction measurements with a high resolution, e.g., >5 Hz, can be carried out in step 200, for example on the head of a patient. Accordingly, a temporal course of an optical density OD(t) for preferably several components in the considered measuring volume can be determined as a negative decadic logarithm of the quotient of the intensity of the detected near-infrared radiation and the intensity of the emitted near-infrared radiation. Here, optical densities of the tissue under consideration can be determined for at least the components hemoglobin, deoxyhemoglobin, water, background, and/or indicator. In step 210, a preferably calibratable system matrix can be generated. For this purpose, components of the background of the measurement volume and their proportions as well as their influence on extinction coefficients of the background components are taken into account. Thus, using literature values and/or comparative measurements as well as considering the density of the organ, absolute concentration values of components in the considered measuring volume can be determined in step 220, in particular of hemoglobin, deoxyhemoglobin, background and/or water as well as of the injected indicator, as indicated in step 230. Such determined values also may be adjusted to patient specific values, e.g., adjusting bone density based on age, or accounting for skin tone.

In step 240, the evaluation algorithm subjects the changes in concentration over time of the indicator determined in step 230 cICG(t), to a plausibility check. If this assessment fails, the measurement is considered to be invalid. In step 250, the time course of the concentration of the indicator. cICG(t) is fitted using a lognormal function. At step 260, mean transit time mtt is estimated, which lies in a determinable range between definable limit values, i.e., minimum transit time mttₘᵢₙ and maximum transit time mttₘₐₓ and thus in a definable search interval. The search interval is defined in step 270. A query whether mean transit time mtt is still within the defined range may be made at step 280. Mean transit time mtt is decisive for the iterative determination of inflow function i(t) as well as outflow function o(t), and for determination of the blood volume and/or the blood flow in the organ under consideration. Outflow function o(t) may be determined from the convolution integral of inflow function i(t) and transport function g(t), which in turn can be determined approximately, in particular the ascending branch of the curve, with a lognormal function as a function of mean transit time mtt.

In step 290, corresponding transport function g(t) is determined based on minimum transit time mtt. In step 300, the time course of inflow function i(t) and the time course of outflow function o(t) then may be determined. The time course of inflow function i(t) and the time course of outflow function o(t) may be approximately described using a corresponding lognormal distribution, as indicated in step 310. In step 320, a plausibility check is performed using a plausibility criterion, which defines a termination criterion for the variation of mean transit time mtt.

As a first plausibility criterion, and thus as a termination criterion of the variation of mean transit time mtt, the position of the center of gravity of inflow function i(t) and the center of gravity of outflow function o(t) can be determined, which have a definable distance from each other according to the first plausibility criterion. The definable distance can correspond to mean transit time mtt.

Alternatively, a further or rather second plausibility criterion may be used, which is based on an area to be determined under inflow function i(t) and an area to be determined under outflow function o(t). If the second plausibility criterion is fulfilled, then the areas to be determined are in a definable ratio to each other. Preferably, this ratio is 1:1.

If none of the above plausibility criteria is met, then mean transit time mtt may be varied by an increase of one increment in step 330, for example by 0.5. This increase may continue, and the other steps of the algorithm performed until one of the plausibility criteria is met. Otherwise, such increases are stopped in step 280, if in step 270 the defined maximal allowed value of mtt is reached. In this case, no mtt is determinable, and the measurement is considered to be invalid.

In step 340, blood volume BV and blood flow BF are determined based on the determined mean transit time mtt. To determine the blood volume, the time characteristic of the concentration values of indicator cICG(t) may be described approximately using an exponential function in a time interval t1>0 to t2>t1, and this exponential function then may be extrapolated back to time t=0. Furthermore, the blood volume in the organ tissue under consideration may be regarded as proportional to the areas under inflow function i(t) and outflow function o(t), which can be calculated by integration.

It should be understood that the embodiments described herein are illustrative, and components may be arranged, substituted, combined, and designed in a wide variety of different configurations, all of which are contemplated and fall within the scope of this disclosure. Accordingly, the foregoing description of illustrative embodiments has been presented for purposes of illustration and of description and are not intended to be exhaustive. Rather, it is intended that the scope of the invention be defined by the claims.

1. A method for determining absolute concentration values of components, a blood flow, or a blood volume in tissue of an organ, the method comprising:
emitting radiation having at least one wavelength in a near-infrared spectrum into the tissue and generating, by near-infrared spectroscopy, measurement signals responsive to detected intensities of radiation emerging from the tissue;
converting, using a system matrix and an evaluation algorithm executed on an evaluation unit, a temporal change of the detected intensities of the radiation emerging from the tissue into absolute concentration values of components;
introducing an indicator comprising a dye having an absorption maximum in the near-infrared spectrum and determining a time course of concentration values of the indicator in the tissue;
deriving a mean transit time (mtt) from the time course of concentration values of the indicator and using at least one transport function g(t) that characterizes blood flow in the tissue; and
calculating at least one of blood flow or blood volume at least one of from the time course of concentration values of the indicator or from parameters derived therefrom.

2. The method of claim 1, wherein the system matrix is calibratable using known concentration values of components in the tissue, measurable concentration values of components in healthy tissue or definable boundary conditions for limiting concentration values of components in the tissue.

3. The method of claim 1, wherein the measurement signals correspond to absolute concentration values of one or more of hemoglobin, deoxyhemoglobin, water, background, or the indicator, and enable determination of the blood volume or the blood flow in the tissue.

4. The method of claim 1, wherein the evaluation algorithm is programmed to:
a) accept measurement signals that transmitted to the evaluation unit, the measurement signals based on an emitted and detected portion of emitted radiation with at least one wavelength in the near-infrared spectrum;
b) determine absolute concentration values of components in the tissue of at least hemoglobin, deoxyhemoglobin, background, or water;
c) determine the time course of the concentration of the indicator in the tissue from the measurement signals;
d) iteratively determine an inflow function i(t) and an outflow function o(t) indicative of blood flow in the tissue using the transport function g(t) with the determinable mean transit time (mtt) until a termination criterion is reached;
e) fit the iteratively determined inflow function i(t) and the iteratively determined outflow function o(t) using a lognormal function or another function representing tissue transit system;
f) calculate the blood volume in the tissue using one of the functions determined in steps c) or e); and
g) calculate the blood flow in the tissue as a quotient of the blood volume calculated in step f) and the mean transit time (mtt) determined in step d).

5. The method of claim 4, wherein in step f) the blood volume in the tissue is determinable using an exponential regression analysis of the time course of concentration of the indicator.

6. The method of claim 4, wherein in step f) the blood volume in the tissue is determined from an area under the inflow function i(t) and the outflow function o(t).

7. The method of claim 4, wherein the iterative determination of the inflow function i(t) comprises several steps, wherein in each step an approximation to the inflow function i(t) is calculated according to the formula:
$$i ( t ) = \frac{d}{d t} ( c I CG ( t ) ) + o ( t )$$
where d/dt(cICG(t)) is a determinable temporal change in concentration of the indicator in the tissue, and o(t) is an outflow function determinable from deconvolution of a convolution integral of the inflow function i(t) and the transport function g(t):
$$o ( t ) = i ( t ) * g ( t )$$

8. The method of claim 4, wherein the termination criterion for determining the mean transit time (mtt) is defined by a plausibility criterion for the inflow function i(t) and the outflow function o(t).

9. The method of claim 8, wherein the plausibility criterion is definable as a distance between a centroid of the inflow function i(t) and a centroid of the outflow function o(t) and corresponds to the determinable mean transit time (mtt).

10. The method of claim 8, wherein the plausibility criterion is definable as a ratio of an area under the inflow function i(t) and an area under the outflow function o(t).

11. The method of claim 10, wherein the ratio is 1:1.

12. Apparatus for determining absolute concentration values of components, a blood flow, or a blood volume in tissue of an organ, for use with an indicator introduced into the tissue via an injection device, the indicator comprising a dye having an absorption maximum in the near-infrared spectrum, the system comprising:
a sensor arrangement for emitting radiation having at least one wavelength in a near-infrared spectrum into the tissue and generating, by near-infrared spectroscopy, measurement signals responsive to detected intensities of radiation emerging from the tissue;
an evaluation unit comprising a processor, memory for storing a system matrix, the evaluation unit programmed to execute an evaluation algorithm that:
converts, using a system matrix, a temporal change of the detected intensities of the radiation emerging from the tissue into absolute concentration values of components;
determines a time course of concentration values of the indicator in the tissue;
derives a mean transit time (mtt) from the time course of concentration values of the indicator and using at least one transport function g(t) that characterizes blood flow in the tissue; and
calculates blood flow or blood volume from the time course of concentration values of the indicator or from parameters derived therefrom.

13. The apparatus of claim 12, wherein the system matrix is calibratable using known concentration values of components in the tissue, measurable concentration values of components in healthy tissue or definable boundary conditions for limiting concentration values of components in the tissue.

14. The apparatus of claim 12, wherein the measurement signals correspond to absolute concentration values of one or more of hemoglobin, deoxyhemoglobin, water, background, or the indicator, and wherein the evaluation algorithm enables determination of the blood volume or the blood flow in the tissue.

15. The apparatus of claim 12, wherein the evaluation unit is further programmed to:
a) accept measurement signals based on an emitted and detected portion of emitted radiation with at least one wavelength in the near-infrared spectrum;
b) determine absolute concentration values of components in the tissue of at least hemoglobin, deoxyhemoglobin, background, or water;
c) determine the time course of the concentration of the indicator in the tissue from the measurement signals;
d) iteratively determine an inflow function i(t) and an outflow function o(t) indicative of blood flow in the tissue using a transport function g(t) with a determinable mean transit time (mtt) until a termination criterion is reached;
e) fit an iteratively determined inflow function i(t) and an iteratively determined outflow function o(t) using a lognormal function;
f) calculate the blood volume in the tissue using one of the functions determined in steps c) or e); and
g) calculate the blood flow in the tissue as a quotient of the blood volume calculated in step f) and the mean transit time (mtt) determined in step d).

16. The apparatus of claim 15, wherein the evaluation unit further is programmed to determine the blood volume in the tissue in step f) using an exponential regression analysis of the time course of concentration of the indicator.

17. The apparatus of claim 15, wherein the evaluation unit further is programmed to determine the blood volume in the tissue in step f) from an area under the inflow function i(t) and the outflow function o(t).

18. The apparatus of claim 15, wherein the evaluation unit further is programmed to iteratively determine the inflow function i(t) by several steps, wherein in each step an approximation to the inflow function i(t) is calculated according to the formula:
$$i ( t ) = \frac{d}{d t} ( c I CG ( t ) ) + o ( t )$$
where d/dt(c/CG(t)) is a determinable temporal change in concentration of the indicator in the tissue, and o(t) is an outflow function determinable from deconvolution of a convolution integral of the inflow function i(t) and the transport function g(t):
$$o ( t ) = i ( t ) * g ( t )$$

19. The apparatus of claim 15, wherein the evaluation unit further is programmed so that the termination criterion for determining the mean transit time (mtt) is defined by a plausibility criterion for the inflow function i(t) and the outflow function o(t).

20. The apparatus of claim 19, wherein the evaluation unit further is programmed so that the plausibility criterion is definable as a distance between a centroid of the inflow function i(t) and a centroid of the outflow function o(t) and corresponds to the determinable mean transit time (mtt).

21. The apparatus of claim 19, wherein the evaluation unit further is programmed so that the plausibility criterion is definable as a ratio of an area under the inflow function i(t) and an area under the outflow function o(t).
